(19) United States
(12) Patent Application Publication
(10) Pub. No.: US 2011/0152219 A1
(43) Pub. Date: Jun. 23, 2011

(54) USE OF ENHANCERS, POSSIBLY ASSOCIATED TO RIBOFLAVIN, AS WELL AS CORRESPONDING OPHTHALMIC COMPOSITIONS FOR CORNEAL CROSS-LINKING IN THE TREATMENT OF THE KERATOCONUS OR OF OTHER CORNEAL ECTASIC DISORDERS

(75) Inventors: Marcello STAGNI, CATANIA (IT); Edoardo STAGNI, CATANIA (IT); Massimo FILIPPELLO, CATANIA (IT); Giovanni CAVALLO, ROME (IT); Eugenio SODO, ROME (IT)

(73) Assignee: SOOFT ITALIA SPA, MONTEGIORGIO (Ascoli Piceno) (IT)

(21) Appl. No.: 13/036,492

(22) Filed: Feb. 28, 2011

Related U.S. Application Data
(63) Continuation-in-part of application No. PCT/IT2009/000392, filed on Aug. 27, 2009.

(30) Foreign Application Priority Data
Aug. 28, 2008 (IT) RM2008A000472

(51) Int. Cl. A61K 31/675; A61K 31/525; A61K 31/473; A61K 31/655; A61K 31/5415; A61K 31/352; A61P 27/02

(52) U.S. Cl. 514/81; 514/251; 514/297; 514/150; 514/224.8; 514/454

(57) ABSTRACT

The use of enhancers with possibly riboflavin, as well as the corresponding compositions for the treatment of keratoconus or other ectasic corneal disorders by the method of corneal cross-linking.

The present invention relates to the use of enhancers of absorption for the preparation of ophthalmic compositions (in particular collyriums) possibly containing riboflavin, designed to imbibe the corneal stroma without having to proceed, in order to obtain said imbibition, to the removal of the corneal epithelium (de-epithelization) in the practice of the treatment of keratoconus, or other ectasic corneal disorders, by means of cross-linking, and also relates to the corresponding ophthalmic compositions for the technique of corneal cross-linking.

Keratoconus is a serious disease of the cornea since it is a non-inflammatory progressive dystrophy that each year affects approximately 50 persons in every 100,000, generally young people of between 10 and 20 years of age. Keratoconus is a genetic disease with a higher frequency amongst females and at times appears to be correlated to dysfunctions of endocrine glands (hypophysis and thyroid). It can affect both eyes in approximately 85% of cases and has an evolution that may vary from subject to subject.

Upon onset of this disease, there appears an irregular curvature that modifies the refractive power of the cornea, producing distorsions of images and a confused close and distant vision. The patient complains in any case of a reduction of vision, above all distant vision. The vision continues to regress irreversibly, with a consequent need for frequent change of spectacles, and for this reason it may at first be mistaken for a myopia associated to astigmatism.

On account of the congenital structural weakness of the corneal stroma due to said disease, after some years the cornea progressively tends to wear out and thin out towards the apex. There then occurs an irregular curvature of the cornea, which loses its spherical shape and assumes the characteristic cone shape (keratoconus).

Using the biomicroscope there may be noted a considerable reduction in the corneal thickness at the top of keratoconus. Over time, the top of keratoconus becomes opaque on account of an alteration in the nutriment of that part of the cornea, which in the most acute forms can present a corneal curvature of more than 62D and reach a corneal thickness of even 446 μm.

If the disease is neglected, the top can ulcerate with consequent perforation of the cornea; there appear pain, lacrimation and spasm of the eyelids. These changes of the cornea due to keratoconus produce an alteration in the disposition of the corneal protein, causing micro-scars that further distort the images and in some cases prevent passage of light, thus giving rise to a troublesome dazzling feeling, above all at times of the day when the Sun is low on the horizon (sunrise and sunset).

As already mentioned, in order to correct the visus it becomes necessary to change spectacles frequently. Only after the use of spectacles has proven unsatisfactory, in milder forms rigid contact lenses may be applied.

The real problem arises when the cornea affected by keratoconus undergoes considerable thinning or if cicatrization occurs following upon lacerations of the corneal surface, rendering necessary even surgical transplantation of the cornea (keratoplasty).

In 2002 so-called lamellar keratoplasty was introduced in Italy for the treatment of keratoconus, whereby, in practice, not the entire cornea is replaced, but only the outer thickness, i.e., the part affected by the disease.

However, already by 1997 in Germany, in the ophthalmic clinic of the Carl Gustaw Carus University of Dresda, a new safer and less invasive technique was developed, referred to as “corneal cross-linking”, which uses in particular riboflavin, activated by a UV laser; in 2005 this technique was tested also in Italy and is by now widely used successfully in various Italian eye clinics.

Corneal cross-linking is a low-invasive method, which uses riboflavin activated by a UV laser (366 nm); said method is painless and is carried out in day-hospital. Cross-linking enables reinforcement of the structure of the cornea affected by keratoconus through the interweaving and increase in links (cross-linking) between the fibres of the corneal collagen. From the clinical studies conducted, this method has proved able to reduce the astigmatism associated to keratoconus as well as to slow down or arrest evolution of keratoconus, thus avoiding the need for transplantation of the cornea. Also other disorders characterized by corneal ecstasia benefit from treatment using the cross-linking method.

Corneal cross-linking is carried out by applying a local corneal anaesthesia for making the abrasion of the corneal epithelium (de-epithelization) having a diameter of 8-9 mm. This is followed by a frequent instillation of a 0.1% riboflavin-based ophthalmic solution followed, after 15 minutes, by irradiation with ultraviolet (UV-A) emitter for 5 minutes, and then by new instillation and new irradiation for a total of 6 cycles (30 minutes in all).

Riboflavin, in particular riboflavin phosphate, which is commonly used in corneal cross-linking, is a hydrophilic photosensitizing and photopolymerizing molecule with a poor capacity for diffusing through the epithelium and hence reaching the corneal stroma. It is therefore necessary to facilitate absorption thereof and complete imbibition of the corneal stroma before starting the irradiation with UV-A, by means of removal of the corneal epithelium (de-epithelization). This procedure can create, albeit rarely, complications at a corneal level, pain, in addition to being a method that renders the task of the oculist more difficult.

It would hence be desirable to improve the absorption of riboflavin, without having to resort to de-epithelization of the cornea, hence obtaining a non-invasive corneal cross-linking with elimination or reduction of the anaesthesia and consequent fast healing without pain or possible complications.

It would moreover be desirable to envisage compounds that enable ease of epithelial absorption but are provided with equal or higher activity than riboflavin in determining photosensitization and photopolymerization of the collagen fibrils; said compounds should preferably be activated at intensities of light close to the visible in order to overcome the harmful effects of the repeated cycles of UV radiation necessary for activation of riboflavin.

It has now been surprisingly discovered that by administering on the cornea ophthalmic compositions containing enhancers chosen from among:

bio-enhancers (i.e., substances that favour the passage of riboflavin or of other photosensitizing and photopolymerizing substances through the corneal epithelium, enabling absorption by the corneal stroma itself), such as for example EDTA associated to tromethamine, ophthalmologically acceptable EDTA salts associated to tromethamine, polysorbate 80, thromethamine, azone, benzalkonium chloride, cetylpyridinium chloride, cetyltrimethylammonium chloride, lauric acid, menthol, methoxysalicylate, polyoxyethylene, sodium glycholate, sodium glycodeoxycholate, sodium lauryl sulphate, sodium salicylate, sodium taurocholate, sodium taurodeoxycholate; and/or

photo-enhancers (i.e., photosensitive and photopolymerizing substances that can be readily absorbed by the epithelium and that, like riboflavin, can also be activated by light to form corneal cross-linking), such as for example the dyes acridine yellow, quinidine yellow, methylene blue, and erythrosine, the structures of which are given below:

acridine yellow:
useful absorption: 460 nm

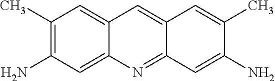

quinidine yellow

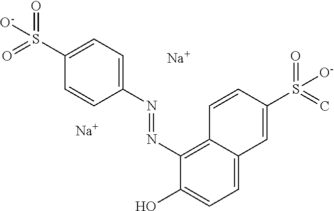

methylene blue
useful absorption: 668 nm

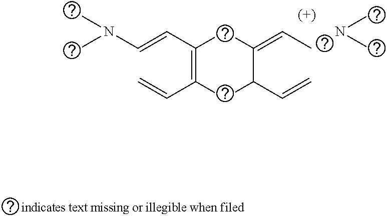

erythrosine B
2′,4′,5′,7′-Tetraiodofluorescein disodium salt

### Absorption: 525 nm

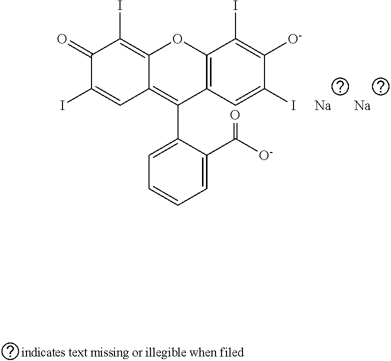

### Molecular Formula:

C₂₀H₆I₄Na₂O₅

### Molecular Weight:

879.86

possibly associated to riboflavin, an improved corneal cross-linking is obtained by means of an irradiation close to the visible that is less harmful, without any need for de-epithelization.

The present invention hence envisages the use of the enhancers described above, either alone or variously mixed together, possibly with riboflavin, for the preparation of ophthalmic compositions to be used for cross-linking in keratoconus or in other corneal ectasias.

The enhancers according to the present invention can be used for application directly on the cornea for cross-linking of keratoconus or other corneal ectasias without proving cytotoxic.

Forming a further subject of the present invention are ophthalmic compositions containing the enhancers mentioned above possibly associated to riboflavin; said ophthalmic composition can be prepared according to known techniques and can contain in particular:
one or more bio-enhancers with one or more photo-enhancers and possibly riboflavin;
one or more bio-enhancers with possibly riboflavin;
one or more photo-enhancers with possibly riboflavin.

After ocular administration of the compositions described above containing just one or more bio-enhancers or else one or more photo-enhancers, there may possibly be applied directly on the cornea a solution of one or more photosensitizing and photopolymerizing substances, in particular riboflavin.

The bio-enhancers of the present invention are present in appropriate amounts in all the compositions described above chosen between 0.001 wt % and 5 wt % with respect to the composition.

EDTA has been widely used for inclusion in ophthalmic formulations. For example, U.S. Pat. No. 5,817,630 to Hofmann et al. describes the incorporation of 0.05 wt. % to 0.5 wt. % EDTA into glutathione eye drops, U.S. Pat. No. 5,283,236 to Chiou describes the use of EDTA as a permeation-enhancing agent for the systemic delivery of polypeptides through the eye, U.S. Pat. No. 6,376,534 to Isaji et al. suggests that EDTA may be effective in inhibiting secondary cataracts, and U.S. Pat. No. 6,348,508 to Denick Jr. et al. describes EDTA as a sequestering agent to bind metal ions. In addition to its use as a chelating agent, EDTA has also been widely used as a preservative in place of benzalkonium chloride, as described, for example, in U.S. Pat. No. 6,211,238 to Castillo et al. U.S. Pat. No. 6,265,444 to Bowman et al. discloses use of EDTA as a preservative and stabilizer. However, EDTA has generally not been applied topically in any significant concentration in ophthalmic formulations because of its poor penetration through the epithelium of the cornea.

The enhancer capability of the present formulation mainly depends on the EDTA and tromethamine association since the two compounds form together a ion-pair between the EDTA non salified carboxyl and the tromethamine which has a notable membrane penetrative capacity.

Tromethamine (Tris(hydroxymethyl)aminomethane) is an amino alcohol widely used in several chemical products such as cosmetics, industrial buffer solutions, components of pharmaceutical compositions; due to its low toxicity it is biologically inert and it is used intracellularly and extracellularly as an alkalizer for the correction of metabolic acidosis. Although widely used, specific clinic letterature reported only one case of periorbital dermatitis induced by a tromethamine containing gel. (Nahas et al. Guidelines for the treatment of acidaemia with THAM. Drugs 1998. 55:191-224). Experimental data have pointed out that, if the tromethamine is preferably present at between 0.05 wt % and 0.5 wt %, and EDTA is preferably present at between 0.05 wt % and 0.5 wt %, the riboflavin absorption is immediate.

The photosensitizing and photopolymerizing substances (photo-enhancers), amongst which riboflavin, of the present invention are used in appropriate amounts chosen between 0.001 wt % and 1 wt % with respect to the composition.

In addition, the riboflavin preferably used in the present invention is riboflavin phosphate in appropriate amounts in all the compositions described above; in particular it is preferably present at between 0.05 wt % and 0.3 wt % of the composition of the present invention.

The ophthalmic compositions of the present invention can be prepared in the technical form of collyriums, eye-drops, eye-washes, ointments, and in any case in all the pharmaceutical technical forms that enable a corneal application according to known techniques; given hereinafter are examples provided by way of illustration, without this implying any limit to the present invention. Other aspects, advantages and modifications within the scope of the invention will be apparent to those skilled in the art to which the invention pertains.

Formulations are reported below, the dosage of the individual components is expressed in weight percentage.

### EXAMPLE 1

PERMEABILIZING SOLUTION 3
Dextran T500
EDTA 0.1% Polysorbate 80 1%
Tromethamine 0.1%

| Ingredients | % w/w |
|---|---|
| DextranT500 | 15 |
| Sodium EDTA | 0.1 |
| Polysorbate 80 1% | 1 |
| Tromethamine | 0.1 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 142 MOs |
| PH | 7.2 |
| Sterilized filtration | Yes |

### EXAMPLE 2

PERMEABILIZING SOLUTION 4
Dextran T500
EDTA 0.1% Tromethamine 0.1

| Ingredients | % w/w |
|---|---|
| Riboflavin phosphate dihydrated sodium salt | 0.147 |
| DextranT500 | 15 |
| Sodium EDTA | 0.1 |
| Tromethamine | 0.1 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 142 MOs |
| PH | 7.2 |
| Sterilized filtration | Yes |

### EXAMPLE 3

PERMEABILIZING SOLUTION 5
Dextran T500
EDTA 0.1% Tromethamine 0.1

| Ingredients | % w/w |
|---|---|
| DextranT500 | 15 |
| Sodium EDTA | 0.2 |
| Tromethamine | 0.2 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 142 MOs |
| PH | 7.2 |
| Sterilized filtration | Yes |

Note that, when the concentration of tromethamine is increased, it is necessary to increase NaH PO₄ sufficiently so as to guarantee a pH of 7-7.2.

### EXAMPLE 4

Preparation E
RIBOFLAVIN PHOSPHATE 0.147 w/w
Dextran T500
Quinoline Yellow 0.050%

| Ingredients | % w/w |
|---|---|
| Riboflavin phosphate dihydrated sodium salt | 0.147 |
| DextranT500 | 20 |
| Quinoline Yellow | 0.050 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 139 mMOs |
| PH | 7.2 |
| Sterilized filtration | Yes |

### EXAMPLE 5

Preparation HYPOTONIC F
RIBOFLAVIN PHOSPHATE 0.147 w/w
Dextran T500
Acridine Yellow 0.050%

| Ingredients | % w/w |
|---|---|
| Riboflavin phosphate dihydrated sodium salt | 0.147 |
| DextranT500 | 20 |
| Acridine Yellow | 0.050 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 139 mMOs |
| PH | 7.1 |
| Sterilized filtration | Yes |

### EXAMPLE 6

Preparation HYPOTONIC G
RIBOFLAVIN PHOSPHATE 0.147 w/w
Dextran T500
ErytrosinB 0.050%

| Ingredients | % w/w |
|---|---|
| Riboflavin phosphate dihydrated sodium salt | 0.147 |
| DextranT500 | 20 |
| Erythrosin B | 0.050 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 139 mMOs |
| PH | 7.1 |
| Sterilized filtration | Yes |

### EXAMPLE 7

Preparation H
RIBOFLAVIN PHOSPHATE 0.147 w/w
Dextran T500
Methylene blue 0.050%

| Ingredients | % w/w |
|---|---|
| Riboflavin phosphate dihydrated sodium salt | 0.147 |
| DextranT500 | 20 |
| Methylene blue | 0.050 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 139 mMOs |
| PH | 7.1 |
| Sterilized filtration | Yes |

### EXAMPLE 8

PERMEABILIZING SOLUTION I
Dextran T500
EDTA 0.1% Tromethamine 0.05%

| Ingredients | % w/w |
|---|---|
| Riboflavin phosphate dihydrated sodium salt | 0.147 |
| Dextran T500 | 15 |
| Sodium EDTA | 0.1 |
| Tromethamine | 0.05 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 142 MOs |
| PH | 7.2 |
| Sterilized by filtration | Yes |

It should be pointed out that the formulations 1-8 may also be isotonic or even hypertonic with the addition of sufficient sodium chloride (or other osmolyte) to achieve said purpose.

Experimental data of the studies carried out by the applicant are presented below.

### Exerimental Study 1

In the present study it has been evaluated the possibility to perform a trans-epithelial CXL using a formulation containing 0.1% riboflavin in association with enhancer enabling riboflavin to permeate corneal stroma even in presence of continuous epithelium.
In the study 20 patients have been enrolled (14 males and 6 females, aged between 12 and 42 years), affected by bilateral progressive keratoconus II and III graded, according to Amsler-Krumeich classification. Only one eye has been subjected to the treatment, while the controlateral one has been used as control. The treatment has been performed in the eye presenting major curvature and minor corneal thickness. All patients presented an average corneal thickness of 412.9 micron (minimal thickness 380, maximal 444). In all patients disease progression has been assessed by increased maximal curvature of cone apex of at least 1 dioptre, topographically measured in the last 6 months, or by a decreased corneal thickness higher than 2% and an increased central corneal astigmatist of 1 dioptre in the last 6 months.
Patients have been checked before the treatment and followed up at 7 days, 15 days, 1, 3, 6, 9 months after the treatment.
Visus has been tested in condition of natural myosis using Log Mar ETDRS charts (Early Treatment Diabetic Retinopathy Study) according to the procedure described by Ferris et al. 1982. Am J. Ophthalmol. 94: 91-6. Visus has been measured at 4 meters distance. Corneas have been analyzed by slit-lamp examination, by ultrasound and optical pachymetry, corneal topography and aberrometry, (Topografo Optikon Keratron Scout software 4.2). Keratoconus evolution has been topographically defined using axial, curvature and refractive maps. Pathology progression has been followed up using propter parameters.
Pachimetry has been performed using ultrasound pachimetry (Optikon Mizar) and corneal OCT (Spectral OCT SLO-OPKO, USA).
Three days before the treatment, norfloxacine has been administered 50 μl (about 1 drop) 4 times/day.
Twenty minutes before the treatment, cornea has been anaesthetized using 0.2% oxybuprocaine hydrochloride, and, in order to reduce damages to other ocular structures due to UV exposition, myosis has been induced by administration of 1% pilocarpine.
The composition according to the present invention containing 0.1% riboflavin in association with enhancer has been administered two hours before the treatment 50 μl (about 1 drop) every 10 minutes.
Then UV irradiation has been performed for 30 minutes at 2.9-3 mW/cm² with a 8 mm diameter spot according to the protocol established by Caporossi et al. 2006. Eur J. Ophthalmol 16:530-5. During the irradiation the amount of composition containing riboflavin has been constantly maintained in contact with the corneal epithelium. Following the treatment one drop of norfloxacine and one artificial eye drop containing 0.15% Hyaluronic acid and amino acids have been added to the eye. Corneas have been checked by slit-lamp to evaluate the integrity of the epithelium.

### Results.

TABLE 1
Natural and corrected visus

| | Pre CXL | | 1 month | | 3 months | | 6 months | | 9 months | |
|---|---|---|---|---|---|---|---|---|---|---|
| | CXL | Controls | CXL | Controls | CXL | Controls | CXL | Controls | CXL | Controls |
| UCVA | 0.71 ± 0.12 | 0.84 ± | 0.49 ± 0.12 | 0.81 ± 0.18 | 0.40 ± 0.15 | 0.80 ± 0.09 | 0.36 ± 0.19 | 0.85 ± 0.10 | 0.36 ± 0.07 | 0.88 ± 0.13 |
| | | 0.23 | | | | | | | | |
| BCVA | 0.35 ± 0.23 | 0.46 ± | 0.26 ± 0.10 | 0.48 ± 0.29 | 0.22 ± 0.08 | 0.50 ± 0.06 | 0.18 ± 0.16 | 0.62 ± 0.08 | 0.16 ± 0.10 | 0.66 ± 0.11 |
| | | 0.21 | | | | | | | | |

Legend:
Pre CXL = visus before cross linking (CXL);
UCVA = natural visus;
BCVA = corrected visus;
LogMar = Logarithm of minimal risolution corner;
p < 0.05.

Table 1 reports visus measurements pre- and post-treatment with the formulation of the present invention containing 0.1% riboflavin in the nine months of follow up period. Natural and corrected visus start to ameliorate in the first month and improvement continues up to sixth month. Amelioration of corrected visus proceeds up to ninth month. Whereas, in the untreated eye was evident a progressive worsening of the natural and corrected visus, which is particularly evident after six months and indicates keratoconus progression.

TABLE 2
Central cheratometric variations (3 mm)

| | Pre CXL | | 1 month | | 3 months | | 6 months | | 9 months | |
|---|---|---|---|---|---|---|---|---|---|---|
| | CXL | Controls | CXL | Controls | CXL | Controls | CXL | Controls | CXL | Controls |
| Sim kS | 51.02 ± 1.10 | 51.12 ± | 49.05 ± 0.92 | 51.10 ± 1.04 | 48.65 ± 0.89 | 51.42 ± 0.96 | 47.82 ± 0.78 | 51.40 ± 0.92 | 47.85 ± 0.71 | 51.32 ± 1.13 |
| | | 1.02 | | | | | | | | |
| Sim kF | 45.13 ± 0.97 | 46.12 ± | 44.46 ± 1.03 | 46.12 ± 0.65 | 44.13 ± 0.89 | 46.52 ± 0.91 | 44.57 ± 1.11 | 46.74 ± 0.71 | 47.85 ± 0.84 | 46.23 ± 0.50 |
| | | 0.99 | | | | | | | | |
| Sim Cyl | 5.89 | 4.91 | 4.59 | 4.98 | 4.55 | 4.91 | 3.32 | 4.70 | 3.55 | 5.12 |

Legend:
SimkS = cheratometry of more curved meridian,
SimkF = cheratometry of more flat meridian,
Sim cyl: corneal cylinder (K1-K2).
p < 0.05.

In table 2 cheratometric data detected by topography before and 1, 3, 6 months after the treatment in a central area of 3 mm.

TABLE 3
Maximal curvature (KcAK) and parameters axial
magnitudo, apical magnitudo

| | Pre CXL | | 1 month | | 3 months | | 6 months | | 9 months | |
|---|---|---|---|---|---|---|---|---|---|---|
| | CXL | Controls | CXL | Controls | CXL | Controls | CXL | Controls | CXL | Controls |
| Kcak | 59.12 ± 1.10 | 58.89 ± | 58.01 ± 0.92 | 58.92 ± 2.34 | 57.42 ± 0.89 | 59.43 ± 1.87 | 57.31 ± 0.78 | 59.86 ± 2.45 | 57.56 ± 1.21 | 60.06 ± 1.13 |
| | | 2.02 | | | | | | | | |
| Mc | 56.46 ± 0.97 | 56.31 ± | 55.73 ± 1.41 | 56.29 ± 2.18 | 55.52 ± 0.89 | 57.02 ± 0.91 | 55.49 ± 1.11 | 57.59 ± 2.02 | 55.51 ± 1.23 | 58.18 ± 0.93 |
| | | 1.93 | | | | | | | | |
| Ma | 23.89 ± 0.75 | 21.91 ± | 20.07 ± 2.42 | 21.98 ± 1.67* | 20.09 ± 2.50 | 23.06 ± 1.4 | 20.01 ± 2.02 | 23.21 ± 0.67 | 20.12 ± 1.22 | 23.81 ± 0.88 |
| | | 2.05 | | | | | | | | |

Legend:
KcAK = Keratoconus apical keratometry,
ma = axial magnitudo,
mc = apical magnitudo.
p < 0.05
*p > 0.05.

Table 3 shows variation of maximal curvature of cone apex (KcAC) and propter parametes used by the topographer software for studying keratoconus progression.

TABLE 4
Corneal aberration

| | Pre CXL | | 1 month | | 3 months | | 6 months | | 9 months | |
|---|---|---|---|---|---|---|---|---|---|---|
| | CXL | Controls | CXL | Controls | CXL | Controls | CXL | Controls | CXL | Controls |
| Rms | 4.68 ± 0.27 | 4.43 ± 0.75 | 4.21 ± 0.66 | 4.12 ± 0.83 | 3.75 ± 0.59 | 4.39 ± 1.47 | 3.01 ± 0.38 | 4.56 ± 2.45 | 3.21 ± 0.45 | 4.71 ± 1.02 |
| Coma | 2.21 ± 0.97 | 2.28 ± 1.93 | 2.19 ± 1.04 | 2.10 ± 1.74 | 1.72 ± 0.32 | 2.23 ± 1.05 | 1.65 ± 1.01 | 2.41 ± 1.88 | 1.59 ± 1.23 | 2.39 ± 0.93 |
| S.A. | 0.98 ± 0.15 | 1.12 ± .052 | 0.77 ± 0.42 | 1.08 ± 0.67 | 0.45 ± 0.59 | 1.26 ± 0.72 | 0.45 ± 0.39 | 1.26 ± 0.47 | 0.35 ± 0.64 | 1.31 ± 0.98 |

Legend:
Rms (root mean square),
S.A. (sferical aberration).
p < 0.05.

In table 4 variations of corneal aberrations are reported. Table shows that root mean square and aberrations ameliorate starting from first up to sixth month; afterwards, they tend to stabilize. Whereas, in the untreated eye aberrations tend to worsening. OCT analysis pre- and post-treatment has shown the thickening of corneal stroma, linear shaped, placed at 100 micron from corneal epithelium underneath Bowman membrane. Such alteration of corneal structure is evident 1 month following the treatment.
Corneas have been transparent all over the follow up period and no haze or sub oedema signs typical of de-epitheliation treatment have been detected. No side effects associated to trans-epithelial treatment in the follow up period.

### Exerimental Study 2

The efficacy of formulation of preparation E (example 4), F (example 5), H (example 7), and I (example 8) has been evaluated in treating nine patients (5 males and 3 females), with an average age of 35 years, all affected by keratoconus referred as at stage 1V according to Amsler-Krumeich classification. All patients have been subjected to: slit-lamp examination, corneal topography, corneal pachymetry, corneal optical coherence tomography (OCT), confocal microscopy, visus examination.

Patients have been divided into three experimental groups, each comprising three patients, homogeneous for corneal thickness and disease severity.

Preparation E has been administrated to Group 1, group 2 has been administrated with preparation F, and preparation H has been administrated to Group 3. Formulations have been administrated to the subjects, one drop (approximately 50 μl) only in the worst eye (according Amsler-Krumeich classification) 10 min for 2 hours. At the end of the administration all subjects have been examined in order to ascertain product penetration into corneal stroma. The assessment of product penetration has been performed by slit-lamp examination. No side effects have been reported by the patients, other than occasionally minor temporary irritation at the time of administering the formulation.

In all cases it has been observed that all three dyes generated micro-crevices into corneal epithelium through whom it is possible seeing the slight colouring of the anterior corneal stroma. In case of acridine and quinoline administration colouring of corneal stroma can be enhanced by using a cobalt filter into the slit lamp, following methylene blue administration no filter is needed since the blue color is clearly evident.

Following this preliminary corneal stroma imbibing procedure, it has proceeded to the corneal cross linking, in particular composition of formulation I:

PERMEABILIZING SOLUTION I
Dextran T500
EDTA 0.1% Tromethamine 0.05%

| Ingredients | % w/w |
|---|---|
| Riboflavin phosphate dihydrated sodium salt | 0.147 |
| Dextran T500 | 15 |
| Sodium EDTA | 0.1 |
| Tromethamine | 0.05 |
| Dihydrated monobasic sodium phosphate | 0.067 |
| Dihydrated dibasic sodium phosphate | 0.285 |
| NaCl | 0.00 |
| Distilled water | Up to 100 g |
| Isotonicity: hypotonic | 142 MOs |
| PH | 7.2 |
| Sterilized by filtration | Yes |

has been administrated to the patients of all groups for 15 minutes and corneas have been irradiated by UV light (3 watt) for 30 minutes. It has been treated the eye presenting the worse condition (i.e. with the steeper corneal curvature, according to Amsler-Krumeich classification). The opposite eye has been used as control. At examination by slit lamp patients corneas appeared with complete epithelium despite the presence of slight cracks and light blue coloured stroma. All subjects have been followed-up and examined every month over a period of six months in order to ascertain the therapeutic effect.

Results

First month: Corneal OCT reveals thickening of corneal stroma underneath the Bowman membrane (approximately 100 micron deep). Such stroma alteration is common to all treatment groups with the same thickness, topography maps after one month from the treatment show a slight improvement of maximal curvature. Such improvement is more marked in quinoline treated group. Less pronounced in subjects that received methylene blue. Cross linking signs have not been detected in the three groups; visus amelioration is not evaluable. Cornea appeared transparent when checked by slit lamp. Second month: Corneal OCT reveals slightly less pronounced thickening of corneal stroma. No differences in the three treatment groups. Topography maps showed further amelioration of disease condition with reduction of maximal curvature and general amelioration of other keratoconus topographic indexes. In particular, it has been reported reduction of corneal astigmatism associated to improvement of natural visus. Astigmatism reduction is greater in quinoline treated group. Confocal microscopy has shown corneal stroma thickening associated to corneal cells activation. Cornea appeared transparent when checked by slit lamp.
Third month: No stroma alteration signs are evident by OCT. Corneal maps have been maintained similar to those obtained two months after the treatment with unchanged corneal astigmatism and natural visus in all patient groups. Confocal microscopy revealed an enhanced activation of keratinocytes and nascent cross linking bridges in first third of cornea in the site of stroma thickening detected by OCT. Cornea appeared transparent when checked by slit lamp.
Fourth month: Increased activation of keratinocytes and further cross linking bridges formation. All other parameters resulted unchanged. Cornea appeared transparent when checked by slit lamp.
Fifth month: Further stroma modification characterized by cross linking bridges formation in anterior stroma approximately at 100-150 micron depth. Amelioration of topographic indexes with astigmatism reduction in quinoline treated patients. Transparent corneal pointed out by slit lamp.
Sixth month: Increased cross linking bridges formation in all treated patients. Transparent cornea.
During the six months follow-up period either no variation of corneal transparency and other side effects have been reported.
The stroma alteration with keratinocytes activation occurred in the examined groups three months after the treatment determined the formation of cross linking bridges. Such linkages are present only in the anterior part of the stroma.
The cross linking effect obtained by the described procedure differs from that provided by riboflavin administration and epithelium removal; in fact, in case of cross linking with de-epithelization, corneal stroma modifications are seen by confocal microscopy only in the inferior third of the cornea, while the superior third is unaffected by the treatment.

The presented experimental data demonstrate that keratoconus treatment by quinoline, acridine, metilene blue mediated CXL is possible avoiding corneal epithelium removal, furthermore, results provided by quinoline treatment are more encouraging than other tested dyes.

Briefly, the presented data unequivocally demonstrate that the formulations according the present invention are effective in limiting keratoconus progression, with no side effects neither during the intervention and after that, and during the follow up period. The recorded treatments without epithelium removal using the described compositions are simple and offer several advantages: absence of complications due to de-epitheliation, maintenance of pre-treatment visus, absence of post-treatment pain, the corneal cross linking of the present invention does not require a surgical room to be performed, possibility to treat patient younger than 10 years.

In fact, in the reported case studies patients went back to their usual activities few hours after the treatment, with an evident amelioration of the main observed parameters in one month. Analysis of corneal stroma obtained by the treatments employing the presented formulations determined the formations of stroma thickness at about 100 micron from the epithelium surface. Such alteration differs from that classically obtained by CXL with de-epitheliation. Hence, the two CXL procedures do not provide overlapping results, but they can be considered complementary.

Such evidences open new perspectives in the keratoconus therapy, as the trans-epithelial technique using the presented formulations does not aim to substitute the currently used protocols, but to complete it, for instance, without de-epitheliation and post-surgical procedures, it will be possible to reinforce the effect of the traditional CXL treatment which did not succeed in totally blocking the keratoconus progression.

Furthermore, the CXL procedures performed using the formulation of the present invention allows to widen the range of treatable patients, as due to the major compliance will be possible to treat very young patients affected by initial keratoconus, slowing down pathology evolution and generally improving their therapeutic expectations.

1-9. (canceled)

10. A method for treating keratoconus or of other ectasic disorders by corneal cross-linking, comprising:
administering to a subject in need thereof an effective amount of an ophthalmic composition comprising:
one or more bio-enhancers in order to favor the passage of the ophthalmic composition to the stroma through the corneal epithelium, said one or more bio-enhancers being selected from the group consisting of: EDTA associated to tromethamine, ophthalmologically acceptable EDTA salts associated to tromethamine, polysorbate 80, tromethamine, azone, benzalkonium chloride, cetylpyridinium chloride, cetyltrimethylammonium chloride, lauric acid, menthol, methoxysalicylate, polyoxyethylene, sodium glycholate, sodium glycodeoxycholate, sodium lauryl sulphate, sodium salicylate, sodium taurocholate, sodium taurodeoxycholate, either alone or mixed together; and/or
one or more photo-enhancers chosen from dyes selected from group consisting of: acridine yellow, quinidine yellow, methylene blue, erithrosine, either alone or mixed together, possibly associated to riboflavin.

11. The method according to claim 10 wherein the bio-enhancers are present in an amount chosen between 0.0001-5 wt % with respect to the composition, in particular EDTA is between 0.001 and 1 wt %, tromethamine is between 0.001 and 2 wt %, and polysorbate 80 is between 0.001 and 5 wt %.

12. The method according to claim 11, wherein EDTA is present in an amount between 0.05 and 0.5 wt % and/or tromethamine in an amount between 0.05 and 0.5 wt %.

13. The method according to claim 10, wherein the photo-enhancers are present in an amount of between 0.001 and 1 wt %, and riboflavin phosphate is possibly present in an amount of between 0.05 and 0.3 wt %.

14. The method according to claim 10, wherein riboflavin phosphate is possibly present in an amount of between 0.05 and 0.3 wt %.

15. An ophthalmic composition for treating keratoconus or other ectasic disorders by corneal cross-linking comprising one or more bio-enhancers in order to favor the passage of the ophthalmic composition to the stroma through the corneal epithelium, with one or more photo-enhancers and possibly riboflavin.

16. The composition according to claim 15, wherein one or more bio-enhancers are present possibly with riboflavin.

17. The composition according to claim 15, wherein one or more photo-enhancers are present possibly with riboflavin.

18. A collyrium for the treatment of keratoconus or of other ectasic disorders by corneal cross-linking, comprising an ophthalmic composition,
wherein said ophthalmic composition comprises:
one or more bio-enhancers in order to favor the passage of the ophthalmic composition to the stroma through the corneal epithelium selected from the group consisting of: EDTA associated to tromethamine, ophthalmologically acceptable EDTA salts associated to tromethamine, polysorbate 80, tromethamine, azone, benzalkonium chloride, cetylpyridinium chloride, cetyltrimethylammonium chloride, lauric acid, menthol, methoxysalicylate, polyoxyethylene, sodium glycholate, sodium glycodeoxycholate, sodium lauryl sulphate, sodium salicylate, sodium taurocholate, sodium taurodeoxycholate, either alone or mixed together; and/or one or more photo-enhancers chosen from dyes selected from group consisting of: acridine yellow, quinidine yellow, methylene blue, erithrosine, either alone or mixed together, possibly associated to riboflavin.
